(19) United States
(12) Patent Application Publication
(10) Pub. No.: US 2025/0235159 A1
(43) Pub. Date: Jul. 24, 2025

(54) MEDICAL PRODUCT AND METHOD FOR PRODUCING A PRODUCT, IN PARTICULAR A MEDICAL PRODUCT

(71) Applicant: B. Braun Melsungen AG, Melsungen (DE)

(72) Inventor: Benjamin Sittkus, Donaueschingen, Pfohren (DE)

(21) Appl. No.: 18/703,507

(22) PCT Filed: Nov. 4, 2022
(86) PCT No.: PCT/EP2022/080818

§ 371 Date: Apr. 22, 2024

(30) Foreign Application Priority Data
Nov. 4, 2021 (DE) 10 2021 212 452.6
Apr. 8, 2022 (DE) 10 2022 203 586.0

(51) Int. Cl. A61B 5/00; A61B 5/02; A61B 5/05; A61L 29/06; A61L 29/10; A61M 25/10

(52) U.S. Cl. CPC A61B 5/6853; A61B 5/02007; A61B 5/05; A61L 29/06; A61L 29/106; A61M 25/1029; A61L 2420/02; A61L 2420/08; A61M 2025/1031

(57) ABSTRACT

A medical device, more particularly a balloon catheter, includes at least one multilayer structure with at least one metal layer and at least one elastomer material layer. A process is used to produce a product, such as the medical device.

### CROSS-REFERENCE TO RELATED APPLICATIONS

This application is the United States national stage entry of International Application No. PCT/EP2022/080818, filed on Nov. 4, 2022, and claims priority to German Application No. 10 2021 212 452.6, filed on Nov. 4, 2021, and to German Application No. 10 2022 203 586.0, filed on Apr. 8, 2022. The contents of International Application No. PCT/EP2022/080818, German Application No. 10 2021 212 452.6, and German Application No. 10 2022 203 586.0 are incorporated by reference herein in their entireties.

### FIELD

The invention relates to a medical device, to a process for producing a product, more particularly a medical device, and to a multilayer structure, more particularly in the form of a sensor unit.

### BACKGROUND

The high incidence of arteriosclerotic disease, which continues to grow, means that advanced intraoperative diagnostics and therapeutics are fundamentally of great social and economic importance. Digitization of regulated medical applications requires a secure database and thus supplementary and independent processes for obtaining information. Different and sometimes redundant sensors contribute thereto. The procedures used to date in arteriosclerotic disease, for example imaging procedures (contrast angiography, Doppler and intravascular ultrasound) for assessing the severity of pathological vascular changes, are limited to vessel diameter analysis and thus to the degree of stenosis. The treatment decision can be improved by finer differentiation, but additional information about mechanical vessel wall properties is needed for this. Whereas modern and complex intravascular ultrasound systems are used particularly in studies to obtain insights into the local vessel structure, simple sensor solutions for routine clinical use that could in particular plug the gaps in information on mechanical tissue properties are not yet available on the market.

### SUMMARY

The object of the invention is to provide a medical device, a process for producing a product, more particularly a medical device, and also a multilayer structure, that address in particular the gap in the market mentioned in the introduction.

This object is achieved by a medical device, by a process for producing a product, and by a multilayer structure. Preferred embodiments of the invention are the subject of the description that follows.

According to a first aspect, the invention relates to a medical device, more particularly in the form of a balloon catheter.

The medical device includes at least one multilayer structure that includes the following layers arranged on top of one another or consists of the following layers arranged on top of one another:
at least one metal layer and
at least one elastomer material layer.

The expression “at least one multilayer structure” may for the purposes of the present invention mean just one multilayer structure or a multiplicity of multilayer structures, i.e. two or more multilayer structures.

The multilayer structure may for the purposes of the present invention be formed more particularly as a laminate-form and/or membrane-form structure, more particularly a metal-polymer laminate composite, preferably a metal-polymer laminate membrane. In addition, the multilayer structure may for the purposes of the present invention be referred to more particularly as a metal-polymer multilayer composite structure, more particularly a metal-polymer bilayer composite structure.

The expression “at least one metal layer” may for the purposes of the present invention mean just one metal layer or a multiplicity of metal layers, i.e. two or more metal layers.

The expression “metal layer” means for the purposes of the present invention a layer that includes at least one metal, more particularly in elemental form or in the form of an alloy, or that consists of at least one metal, more particularly in elemental form or in the form of an alloy.

The expression “at least one elastomer material layer” may for the purposes of the present invention mean just one elastomer material layer or a multiplicity of elastomer material layers, i.e. two or more elastomer material layers.

The expression “elastomer material layer” means for the purposes of the present invention a layer that includes at least one elastomer material, more particularly at least one elastomer, preferably at least one hyperelastic polymer, or that consists of at least one elastomer material, more particularly at least one elastomer, preferably at least one hyperelastic polymer.

The expression “hyperelastic polymer” is for the purposes of the present invention to be understood as meaning a polymer, more particularly an elastomer having hyperelastic properties, more particularly high reversible stretchability.

The at least one metal layer preferably has a varying surface morphology. The varying surface morphology is preferably designed to influence, more particularly to increase or decrease, the sensitivity, more particularly the resistive behavior, to mechanical stress, more particularly stretching.

The invention is based more particularly on the surprising finding that the multilayer structure provided by the invention can be used as a sensor unit in medicine, more particularly for determining tissue elasticity, preferably the elasticity of hollow organs, such as blood vessels in particular.

In one embodiment of the invention, the at least one elastomer material layer includes at least one elastomer material, more particularly hyperelastic material, or consists of an elastomer material, more particularly hyperelastic material, selected from the group consisting of elastomers, thermoplastic elastomers, thermoplastic polyamide elastomers, thermoplastic copolyester elastomers, olefin-based thermoplastic elastomers, thermoplastic styrene block copolymers, urethane-based thermoplastic elastomers, olefin-based thermoplastic vulcanizates, olefin-based crosslinked thermoplastic elastomers, natural rubber vulcanizates, synthetic rubber vulcanizates, styrene-butadiene rubber, butadiene rubber (BR), acrylonitrile-butadiene rubber (NBR), butyl rubber (IIR), ethylene-propylene-diene rubber (EPDM), chloroprene rubber (CR), polyisoprene rubber (IR), polyalkylsiloxanes, polydimethylsiloxane, silicone rubbers, silicone elastomers, methyl silicone, vinyl methyl silicone, phenyl vinyl methyl silicone, phenyl-modified silicone, fluoroalkyl silicone, fluoro vinyl methyl silicone, and mixtures of at least two of the aforementioned elastomer materials.

Preferably, the at least one elastomer material layer includes a polyalkylsiloxane, more particularly polydimethylsiloxane, or consists of polyalkylsiloxane, more particularly polydimethylsiloxane.

In addition, the at least one elastomer material layer may more particularly be formed as a film.

More particularly, the at least one elastomer material layer may be formed, more particularly directly or indirectly, on a surface, more particularly an outer and/or inner surface, preferably outer surface, of the medical device.

In a further embodiment of the invention, the at least one elastomer material layer has a layer thickness of 0.0001 mm to 0.2 mm, more particularly 0.0005 mm to 0.1 mm, preferably 0.001 mm to 0.05 mm.

In a further embodiment of the invention, the at least one metal layer includes at least one metal, more particularly in elemental form or in the form of an alloy, or consists of at least one metal, more particularly in elemental form or in the form of an alloy, selected from the group consisting of gold, platinum, indium, tin, copper, silver, gallium and mixtures, more particularly alloys, of at least two of the aforementioned metals.

In addition, the at least one metal layer may more particularly be formed as a film.

In addition, the at least one metal layer may be in serpentine form at least in sections or regions, more particularly only in sections or regions or in a continuous manner, and cover or coat the at least one elastomer material layer.

Preferably, the layer thickness of the at least one metal layer is less than the layer thickness of the at least one elastomer material layer.

In a further embodiment of the invention, the at least one metal layer has a layer thickness of ≤150 nm, more particularly 10 nm to 100 nm, preferably 40 nm to 80 nm.

In a further embodiment of the invention, the at least one elastomer material layer is covered or coated by the at least one metal layer, more particularly directly or indirectly. In addition, the at least one elastomer material layer may in principle be covered or coated by the at least one metal layer completely, i.e. continuously or over the entire surface area, or only partially, i.e. in sections or regions. However, the at least one elastomer material layer is preferably only partially covered or coated by the at least one metal layer. Particularly preferably, the at least one elastomer material layer is directly covered or coated by the at least one metal layer and in sections or regions only.

In a further embodiment of the invention, at least one adhesion layer is arranged or formed between the at least one metal layer and the at least one elastomer material layer.

The expression “at least one adhesion layer” may for the purposes of the present invention mean just one adhesion layer or a multiplicity of adhesion layers, i.e. two or more adhesion layers.

The expression “adhesion layer” means for the purposes of the present invention a layer that includes at least one material or consists of at least one material that is able to permit and/or improve adhesion of the at least one metal layer to the at least one elastomer material layer.

In addition, the at least one adhesion layer may directly or indirectly cover or coat the at least one elastomer material layer. More particularly, the at least one adhesion layer may cover or coat the at least one elastomer material layer completely, i.e. continuously or over the entire surface area, or only partially, i.e. in sections or regions. However, the at least one adhesion layer preferably covers the at least one elastomer material layer only partially.

In addition, the at least one metal layer may directly or indirectly cover or coat the at least one adhesion layer. More particularly, the at least one metal layer may cover or coat the at least one adhesion layer completely, i.e. continuously or over the entire surface area, or only partially, i.e. in sections or regions. However, the at least one metal layer preferably covers the at least one adhesion layer completely.

In a further embodiment of the invention, the at least one adhesion layer is covered or coated by the at least one metal layer, more particularly directly and preferably completely, and the at least one elastomer material layer is covered or coated by the at least one adhesion layer, more particularly directly and preferably only in sections or regions.

In a further embodiment of the invention, the at least one adhesion layer includes at least one material or consists of at least one material selected from the group consisting of titanium, aluminum, chromium, and mixtures of at least two of the aforementioned materials.

In addition, the at least one adhesion layer may more particularly be formed as a film.

In addition, the at least one adhesion layer may be in serpentine form at least in sections or regions, more particularly only in sections or regions or in a continuous manner, and cover or coat the at least one elastomer material layer.

Preferably, the layer thickness of the at least one adhesion layer is less than the layer thickness of the at least one elastomer material layer.

More particularly, the layer thickness of the at least one adhesion layer may be less than the layer thickness of the at least one metal layer.

Preferably, the at least one adhesion layer has a layer thickness of ≤20 nm, more particularly 3 nm to 20 nm, preferably 4 nm to 10 nm.

In a further embodiment of the invention, the at least one metal layer and/or the at least one adhesion layer is/are formed as conductor track(s). The conductor track/tracks are set up to conduct away an electrical measurement signal. For evaluation of the electrical measurement signal, the at least one metal layer and/or the at least one adhesion layer may in this embodiment of the invention be contacted or contactable through electrical conduction with an evaluation unit for evaluating the electrical measurement signal.

In a further embodiment of the invention, the at least one multilayer structure also includes at least one additional elastomer material layer, i.e. just one additional elastomer material layer, or a multiplicity of, i.e. two or more, elastomer material layers. Preferably, the at least one metal layer is covered or coated by the at least one additional elastomer material layer, more particularly directly or indirectly, preferably directly.

Preferably, the layer thickness of the at least one metal layer is less than the layer thickness of the at least one additional elastomer material layer.

Further preferably, the layer thickness of the at least one adhesion layer is less than the layer thickness of the at least one additional elastomer material layer.

The at least one additional elastomer material layer may have a layer thickness of 0.001 mm to 0.15 mm, more particularly 0.001 mm to 0.1 mm, preferably 0.001 mm to 0.05 mm.

In addition, the at least one additional elastomer material layer may more particularly be formed as a film.

In addition, the at least one additional elastomer material layer and the at least one elastomer material layer may be identical or different in form, particularly with regard to the elastomer material and/or the layer thickness.

In addition, the at least one additional elastomer material layer may, more particularly independently of the at least one elastomer material layer, include an elastomer material, more particularly hyperelastic material, or consist of an elastomer material, more particularly hyperelastic material, selected from the group consisting of elastomers, thermoplastic elastomers, thermoplastic polyamide elastomers, thermoplastic copolyester elastomers, olefin-based thermoplastic elastomers, thermoplastic styrene block copolymers, urethane-based thermoplastic elastomers, olefin-based thermoplastic vulcanizates, olefin-based crosslinked thermoplastic elastomers, natural rubber vulcanizates, synthetic rubber vulcanizates, styrene-butadiene rubber, butadiene rubber (BR), acrylonitrile-butadiene rubber (NBR), butyl rubber (IIR), ethylene-propylene-diene rubber (EPDM), chloroprene rubber (CR), polyisoprene rubber (IR), polyalkylsiloxanes, polydimethylsiloxane, silicone rubbers, silicone elastomers, methyl silicone, vinyl methyl silicone, phenyl vinyl methyl silicone, phenyl-modified silicone, fluoroalkyl silicone, fluoro vinyl methyl silicone, and mixtures of at least two of the aforementioned elastomer materials.

Preferably, the at least one additional elastomer material layer includes a polyalkylsiloxane, more particularly polydimethylsiloxane, or consists of polyalkylsiloxane, more particularly polydimethylsiloxane.

In a further embodiment of the invention, the at least one multilayer structure or the at least one metal layer has a varying surface morphology. More particularly, the at least one multilayer structure or the at least one metal layer may have corrugated surface regions and/or non-corrugated surface regions. Preferably, the at least one multilayer structure or the at least one metal layer has surface regions, more particularly corrugated surface regions and/or non-corrugated surface regions, with cracks, more particularly microcracks, and/or surface regions, more particularly corrugated surface regions and/or non-corrugated surface regions, without cracks. Particularly preferably, the at least one multilayer structure or the at least one metal layer has surface regions, more particularly corrugated and/or non-corrugated surface regions, with cracks, more particularly microcracks, and corrugated surface regions without cracks. A varying surface morphology advantageously allows the sensitivity of the at least one metal layer to mechanical stress, more particularly stretching, to be selectively influenced, more particularly increased or decreased.

The expression “microcracks” is for the purposes of the present invention to be understood as meaning cracks having at least one dimension, selected more particularly from the group consisting of length, width, depth, and combinations of at least two of the aforementioned dimensions, in the micrometer and/or nanometer range, more particularly within a range from 1 nm to 5 μm, more particularly 10 nm to 2 μm, preferably 20 nm to 1 μm. The formation of the cracks is preferably dependent on applied coating parameters and/or on pretreatment of the surface of the at least one elastomer material layer.

The expression “corrugated surface regions without cracks” is for the purposes of the present invention to be understood as meaning intermittently repeating corrugated deformations of the surface of the at least one metal layer preferably produced by compressive stress within the at least one metal layer, more particularly by deformation of the underlying at least one elastomer material layer. Preferably, the corrugated surface regions without cracks have at least one dimension, selected more particularly from the group consisting of period length and amplitude and combinations of the two aforementioned dimensions, in the micrometer and/or nanometer range, more particularly within a range from 10 nm to 15 μm, more particularly 20 nm to 10 μm, preferably 40 nm to 6 μm.

In a further embodiment of the invention, the at least one multilayer structure is part of a surface, more particularly an outer and/or inner surface, of the medical device. More particularly, the medical device, more particularly an outer and/or inner surface of the medical device, may be coated with the at least one multilayer structure, preferably at least in sections or regions.

In a further embodiment of the invention, the at least one multilayer structure is part of a wall of the medical device or integrated in a wall of the medical device or formed within a wall of the medical device.

In addition, the at least one multilayer structure may in principle be formed in the longitudinal and/or transverse or circumferential direction of the medical device. Preferably, the at least one multilayer structure is formed in the circumferential direction of the medical device.

In addition, the at least one multilayer structure may be formed as a strip.

More particularly, the medical device may have a multiplicity of, more particularly strip-form, multilayer structures formed in the longitudinal and/or transverse or circumferential direction of the medical device.

In a further embodiment of the invention, the medical device is curved, more particularly cylindrically curved, in form.

Preferably, the medical device is cylindrical, more particularly a hollow cylinder, or tubular in form.

In a further embodiment of the invention, the medical device takes the form of a catheter, preferably balloon catheter.

The expression “balloon catheter” is for the purposes of the present invention to be understood as meaning a catheter, more particularly a catheter made of plastic, rubber, silicone, metal or glass, that is provided with a balloon. The balloon can be inflated, i.e. expanded, either with compressed air or with a liquid.

For example, the catheter may take the form of an angiography catheter, embolectomy catheter, cardiac catheter, peripheral venous catheter, central venous catheter, Broviac catheter, Fogarty catheter, Hickman catheter, Swan-Ganz catheter, endotracheal catheter, bronchial catheter, bladder catheter, nephrostomy catheter, ureter catheter, enterostomy catheter, Shaldon catheter, Demers catheter, balloon irrigation catheter, flow-directed catheter, pigtail catheter or double J catheter.

The catheter particularly preferably takes the form of a balloon catheter, more particularly a plastic catheter bearing at its tip a balloon (occlusion balloon) that can be inflated with compressed air or liquid. The balloon catheter may for example be designed for angioplasty, more particularly percutaneous transluminal coronary angioplasty (PTCA), for embolectomy, valvuloplasty, more particularly in heart valve stenosis, catheterization of the urinary bladder or for bronchial blockade or dilatation.

Particularly preferably, the medical device takes the form of a balloon catheter in which the balloon of the balloon catheter includes the at least one multilayer structure.

In a further embodiment of the invention, the at least one multilayer structure defines at least one sensor unit, more particularly at least one tactile sensor unit. Preferably, the at least one sensor unit takes the form of at least one sensor stack, more particularly a sensor stack having a layered structure. The at least one sensor unit is preferably set up for recording a measured variable, more particularly a physical measured variable, and for converting the same into a measurement signal, preferably electrical measurement signal. The measured variable may more particularly be a mechanical property, for example stretchability or elasticity, or a structural and/or functional parameter of a tissue, more particularly a hollow organ, preferably a blood vessel, more particularly an artery. Alternatively or in combination, the measured variable may be a temperature, a hydrostatic pressure or any other measured variable, more particularly a variable of therapeutic and/or diagnostic interest.

Preferably, the at least one sensor unit is configured for recording a measured variable, for example stretchability or elasticity, that characterizes a blood vessel, more particularly an artery. This advantageously permits the optimization of diagnostics, more particularly intraoperative diagnostics, and/or therapy of vascular disease, more particularly arteriosclerotic disease.

If the medical device takes the form of, for example, a balloon catheter and if the at least one multilayer structure forms part of the balloon of the balloon catheter, it is for example advantageously possible to determine, particularly to determine in a tactile manner, the elasticity of a blood vessel, more particularly of an artery, by recording via the sensor unit a resistance, more particularly electrical resistance, which correlates with an expansion/inflation of the balloon of the balloon catheter and with the pressure used to expand/inflate the balloon of the balloon catheter and more particularly increases reversibly in a linear and/or nonlinear manner, and can be interpreted more particularly by comparing with a reference behavior without contact with surrounding tissue.

According to a second aspect, the invention relates to a process for producing a product, preferably medical device, more particularly according to the first aspect of the invention. The product may more particularly be formed as a metal-polymer laminate composite, more particularly a metal-polymer laminate membrane, more particularly thin metal-polymer laminate membrane. The process includes the following steps:
a) producing at least one multilayer structure having at least one metal layer, more particularly having varying surface morphology, and at least one elastomer material layer and
b) transferring the at least one multilayer structure to a substrate, with the formation of a substrate provided with the at least one multilayer structure.

Preferably, step b) is carried out such that no relevant changes in the surface morphology of the at least one metal layer occur, particularly before the use of the substrate as a medical device.

The substrate used may in principle be a planar, i.e. level or flat, substrate, but preference is given to using a non-planar, more particularly curved, substrate. Preference is given to using a cylindrical or cylindrically curved substrate, more particularly a substrate that is a hollow cylinder or tubular, or a substrate that is conical in form. Preferably, the substrate is a precursor, more particularly a blank, a component such as a part or a semifinished product, of a medical device.

When carrying out step a), the at least one multilayer structure is preferably produced on a transfer film, more particularly semi-compliant transfer film, the transfer film preferably being applied to a carrier. In other words, the at least one multilayer structure is preferably produced on a carrier functionalized or coated with a transfer film. Preferably, the transfer film is removable from the carrier, more particularly detachable. The carrier used may for example be a wafer, more particularly in the form of a circular or rectangular, more particularly rectangular or square, slice. A suitable wafer may be produced from a monocrystalline or polycrystalline blank (an “ingot”). In addition, the wafer may include monocrystalline silicon and/or another material selected for example from the group consisting of silicon carbide, gallium arsenide, indium phosphide, and mixtures of at least two of the aforementioned materials, or may consist of such a material/such materials. In addition, the wafer may have a thickness of for example 0.5 mm. As a transfer film it is for example possible to use a film having an adhesive layer or side that is for example acrylic- and/or methacrylic-based. Preferably, a reduction in the adhesiveness of the adhesive layer or side is brought about by exposure to UV light, as a result of which the carrier film can advantageously be more easily detached from the carrier, but the adhesion to the carrier remains strong enough to avoid premature detachment of the carrier film during further process steps. In addition to the adhesive layer, the transfer film may include a further layer, more particularly a non-adhesive layer, for example of polyethylene terephthalate. The transfer film may for example be applied onto the substrate with a layer thickness of 40 μm to 240 μm, more particularly 60 μm to 155 μm, preferably 75 μm to 120 μm. Preferably, the substrate is coated on one side with the transfer film, more particularly adhesively covered.

Preferably, the at least one elastomer material layer is applied onto the transfer film when performing step a), more particularly by spin coating. In principle, the at least one elastomer material layer may be applied directly or indirectly onto the transfer film, but preferably after exposure of the transfer film to UV to reduce adhesion, so as to avoid contact of the at least one elastomer material layer with UV light and associated possible degradation of the at least one elastomer material layer. Preferably, the at least one elastomer material layer is applied directly onto the transfer film. In addition, the at least one elastomer material layer may be applied onto the transfer film only partially, i.e. only in sections or regions, or continuously, i.e. over the entire surface area. Preferably, the at least one elastomer material layer is applied continuously onto the transfer film. Preferably, the at least one metal layer is then applied onto the at least one elastomer material layer. In principle, the at least one metal layer may be applied directly or indirectly onto the at least one elastomer material layer. In addition, the at least one metal layer may be applied onto the at least one elastomer material layer only partially, i.e. only in sections or regions, or continuously, i.e. over the entire surface area. Preferably, the at least one metal layer is applied onto the at least one elastomer material layer continuously. When carrying out the spin coating, the elastomer material layer or, more precisely, the elastomer material or a precursor thereof, more particularly in the form of a curable prepolymer (preferably in the form of a viscous, curable prepolymer without air inclusions), can be spin-coated onto the transfer film for example at a speed of 400 rpm (revolutions per minute) to 3000 rpm, more particularly 500 rpm to 2000 rpm, preferably 600 rpm to 900 rpm. In addition, the spin coating may be carried out for a period of 30 s to 120 s, more particularly 45 s to 90 s, preferably 60 s to 80 s. The spin coating can in principle be carried out at varying speeds and for varying periods. This can advantageously improve the uniformity of the resulting at least one elastomer material layer. Particularly at high spin speeds, the acceleration in rpm/s (revolutions per minute per second) may also be varied up to a desired spin speed. This ensures uniform flow behavior and uniform wetting after initial application of the elastomer material precursor, more particularly the prepolymer. The acceleration may be varied here from 100 rpm/s to 1000 rpm/s, more particularly from 150 rpm/s to 800 rpm/s, preferably from 200 rpm/s to 400 rpm/s. A vacuum or reduced pressure is then preferably applied, for example for a period of from 10 min to 20 min. This can advantageously result in the formation of a defect-free and preferably even elastomer material layer, more particularly in the form of a membrane. The elastomer material layer can then be cured, for example at a temperature of from 25° C. to 120° C. and more particularly for a period of 30 min to 48 h. The curing of the elastomer material layer can preferably be carried out in an oven.

Preferably, the at least one metal layer is applied onto the at least one elastomer material layer, more particularly directly or indirectly, by a process that structures the surface of the at least one elastomer material layer, more particularly by photolithography, and by a subsequently performed vacuum-based coating process, more particularly by physical vapor deposition, preferably by thermal evaporation.

Preferably, at least one adhesion layer is first applied onto the at least one elastomer material layer by a process that structures the surface of the at least one elastomer material layer, more particularly by photolithography, and by a subsequently performed vacuum-based coating process, more particularly by physical vapor deposition, preferably by thermal evaporation, at least in sections or regions, more particularly only in sections or regions or continuously, i.e. over the entire surface area, more particularly directly or indirectly, and then the at least one metal layer is applied onto the at least one adhesion layer by a process that structures the surface of the at least one elastomer material layer, more particularly by photolithography, and by a subsequently performed vacuum-based coating process, more particularly by physical vapor deposition, more particularly directly or indirectly, preferably directly and while maintaining the vacuum or the reduced pressure, more particularly using reactive metals to form the at least one adhesion layer.

For carrying out the photolithography, a photoresist is preferably first applied onto the at least one elastomer material layer. The photoresist used may in principle be either a positive or negative resist. A positive resist is understood as meaning a photoresist that is removed in the altered, more particularly light-exposed, regions. A negative resist is understood as meaning a photoresist that is removed in the unaltered, more particularly unexposed, regions. Preference is given to using a negative resist as the photoresist. Preferably, the photoresist is applied onto the at least one elastomer material layer by spin coating. The spin coating can be carried out for example at a speed of 1000 rpm to 4000 rpm, more particularly 2000 rpm to 3000 rpm, and more particularly for a period of 45 s to 90 s, preferably 60 s to 75 s. The photoresist applied onto the at least one elastomer material layer can then be heated, more particularly at a temperature of 90° C. to 110° C. and more particularly for a period of 90 s to 2 min. This results in the desorption of solvents present in the photoresist, more particularly propylene glycol monomethyl ether acetate (PGMEA), thereby stabilizing the photoresist. A photo mask is then laid onto the photoresist applied onto the at least one elastomer material layer. The photo mask used may in principle be a mask consisting of—more particularly high-purity—quartz glass or calcium fluoride. The photo mask may also be provided, for example on one side, with a chrome layer, more particularly a structured chrome layer.

As a next step when performing the photolithography, an exposure to light is preferably carried out. This advantageously transfers the image of the photo mask onto the photoresist. This results in a lithographic mask that more particularly allows further processing, preferably by chemical and/or physical processes. The exposure to light can be carried out with a radiation dose of 50 mJ/cm² to 150 mJ/cm², more particularly 60 mJ/cm² to 120 mJ/cm², preferably 70 mJ/cm² to 100 mJ/cm². The radiation source used may be for example a mercury vapor lamp, more particularly a high-pressure mercury vapor lamp. A high-pressure mercury vapor lamp is understood as meaning a mercury vapor lamp having an operating pressure of up to about 1 MPa, which it reaches after warming up for a few minutes. The light used for exposing the photoresist may more particularly have a wavelength of 280 nm to 700 nm, more particularly 300 nm to 500 nm, preferably 350 nm to 450 nm.

After the exposure to light it is possible to carry out a heating step, more particularly a heating step at a temperature of 100° C. to 180° C., preferably 100° C. to 120° C., and more particularly for a period of from 2 min 30 s to 3 min 30 s, preferably 2 min 50 s to 3 min.

After the heating step, the light-exposed photoresist together with the carrier, the transfer film, and the at least one elastomer material layer can be transferred directly to a preheated oven. The oven is preferably preheated to a temperature lower than the temperature in the heating step described in the previous paragraph. Preferably, the oven is preheated to a temperature of 100° C.

After the abovementioned heating step and more particularly before the photoresist is developed as described below, preferably after switching off the abovementioned oven, the photoresist can be cooled, more particularly to a temperature of 18° C. to 30° C., preferably 20° C. to 25° C. In addition, the photoresist can be cooled, more particularly for a period of 5 h. This can advantageously prevent the formation of cracks within the photoresist.

Finally, when carrying out the photolithography the photoresist preferably undergoes development. This essentially results in the dissolution of the light-exposed regions of the photoresist or, more particularly when the photoresist cures on exposure to light, in the dissolution of the unexposed regions of the photoresist. Preference is given to using a developer solution to develop the photoresist. This allows the soluble regions of the photoresist to be dissolved, for example by wet-chemical spraying, dipping or dripping, and then removed. The developer solution used may for example be an aqueous solution containing tetramethylammonium hydroxide (TMAH). Alternatively, a developer solution based on buffered sodium hydroxide solution or buffered potassium hydroxide solution or based on sodium phosphate and sodium metasilicate can be used. The development of the photoresist can for example be carried out for a period of 1 min 30 s to 2 min 20 s, more particularly 1 min 50 s to 2 min 10 s. This can then be followed by rinsing with water, more particularly deionized water, and drying under inert gas, more particularly nitrogen.

Alternatively, structuring of the surface of the at least one elastomer material layer can be carried out using an aperture mask or stencil mask, for example a mask including or consisting of nickel or a nickel-phosphorus alloy, followed by exposure to UV radiation.

In order to be able to influence a varying surface morphology after deposition of the at least one metal layer with or without at least one adhesion layer in different regions, it is possible to carry out a selective plasma treatment of the surface of the elastomer material layer, more particularly before the introduction of the elastomer material layer into a coating chamber. For this, the photolithographic masking carried out for this purpose and/or the corresponding stencil mask may be designed so as to expose only parts of the surfaces to be coated later with at least one metal layer and optionally with the at least one adhesion layer. Introduction into a plasma chamber and exposure of the exposed surfaces to a plasma allows the surface properties of the elastomer material surface to be altered, thereby influencing the surface morphology of the at least one metal layer in these regions that is formed later during deposition. The influence of the plasma on the surface of the at least one elastomer material layer depends also on the choice of process gas and on the process parameters. In order to influence layer growth such that a corrugated closed surface forms, oxygen in particular can be used as a process gas, preferably at plasma powers of 50 W to 150 W, a process pressure of 0.5 mbar to 1.5 mbar, and a process gas flow rate of from 100 sccm (standard cubic centimeters per minute) to 700 sccm, preferably from 300 sccm to 500 sccm. The duration of plasma treatment may be 5 s to 40 s, more particularly 10 s to 35 s, preferably 15 s to 30 s.

The physical vapor deposition may be selected from the group consisting of thermal evaporation, electron beam evaporation, laser beam evaporation, arc evaporation, molecular beam epitaxy, sputtering, ion beam-assisted deposition, ion plating, ICB technique, and a combination of at least two of the aforementioned physical vapor depositions. In physical vapor deposition, the material to be deposited is generally present in solid form in a largely evacuated coating chamber. As a result of being bombarded with laser beams, magnetically deflected ions or electrons, of arc discharge, or of heating (more particularly heating to close to the boiling point), the material, which can also be referred to as the target, undergoes evaporation. The evaporated material moves through the coating chamber either ballistically or under guidance by electric fields and in doing so hits the parts to be coated, where a layer forms. In order that the evaporated particles actually reach the parts to be coated and are not lost through scattering by gas particles, the operation is usually carried out under reduced pressure. Typical working pressures are in the range from 10⁻⁴ Pa to 10 Pa.

After applying the at least one metal layer onto the at least one adhesion layer and/or onto the at least one elastomer material layer, the photo mask, consisting of the unexposed regions of the photoresist, is preferably removed. Preference is given to removing the unexposed regions of the photoresist with an organic solvent, for example acetone.

When using an aperture mask or stencil mask for structuring the surface of the at least one elastomer material layer, the mask is preferably removed after applying the at least one metal layer onto the at least one adhesion layer and/or at least one elastomer material layer, for example by being pulled off.

When carrying out step a), it is preferable that at least one additional elastomer material layer is applied onto the at least one metal layer, more particularly by spin coating. With regard to further features and advantages, particularly in relation to the at least one additional elastomer material layer and the spin coating, reference is made in full to the above description. The features and advantages described therein, particularly in relation to the at least one elastomer material layer, also apply mutatis mutandis to the at least one additional elastomer material layer.

Various contacting techniques can be employed for contacting the at least one metal layer with the at least one adhesion layer between the at least one elastomer material layer and the at least one additional elastomer material layer. For example, contacting can take place in predefined pockets within the at least one additional elastomer material layer, in which, for example by means of conductive, viscous pastes, an electrically conductive connection is created between defined contact pads of the at least one metal layer with the at least one adhesion layer and leads that lead to an evaluation periphery and that are embedded in the at least one additional elastomer material layer.

Further preferably, before carrying out step b), the at least one multilayer structure together with the transfer film is removed, more particularly detached, from the carrier.

Further preferably, before carrying out step b), the substrate is applied onto a core, with the formation of a cylindrical-, tubular- or conical-shaped substrate. The core is for this purpose usefully cylindrical-, tubular- or conical-shaped. More particularly, the core may be stamp-shaped. In addition, the core may include a metal or consist of a metal. The metal may be for example aluminum.

Preferably, the cylindrical-, tubular- or conical-shaped substrate is detached from the core and the core replaced by a water-soluble material, more particularly a water-soluble wax. In other words, the former core is preferably replaced by a water-soluble core, more particularly a core made of a water-soluble wax. The water-soluble wax may have a melting point of 50° C. to 60° C., more particularly 55° C. to 60° C. Preferably, the detachment of the cylindrical-, tubular- or conical-shaped substrate from the core and/or the replacement of the core by the water-soluble material is carried out in the interior of a forming tool or casting mold, more particularly an openable, multi-part, preferably two-part, forming tool or casting mold. Preferably, the forming tool or casting mold, particularly when in an assembled or closed state, has a shape that corresponds to the shape of the product to be produced.

Preferably, the at least one multilayer structure is then transferred to the cylindrical-, tubular- or conical-shaped substrate. This step is preferably carried out outside the forming tool or casting mold.

The water-soluble material is then removed, more particularly by rinsing with water. This step can in turn preferably be carried out in the forming tool or casting mold. This advantageously allows undesirable stresses on the at least one multilayer structure to be avoided.

When carrying out step b), the at least one multilayer structure is preferably applied onto the substrate using the transfer film. Preferably, the transfer film is removed after forming a connection, preferably a material connection, between the at least one multilayer structure, more particularly the at least one elastomer material layer and/or at least one additional elastomer material layer of the at least one multilayer structure, and the substrate. The connection may be effected for example through curing or crosslinking of the elastomer material or of a precursor, more particularly a prepolymer, thereof and based more particularly on the formation of covalent bonds. Alternatively or in combination, the connection may be based on the formation of non-covalent bonds, more particularly selected from the group consisting of Van der Waals forces, hydrogen bonds, ionic bonds, coordinate bonds, and a combination of at least two of the aforementioned non-covalent bonds. In addition, the connection between the at least one multilayer structure, more particularly the at least one elastomer material layer and/or at least one additional elastomer material layer of the at least one multilayer structure, and the substrate may be formed in a forming tool or casting mold, more particularly in the forming tool or casting mold already mentioned. For this purpose, the substrate provided with the at least one multilayer structure may remain in the forming tool or casting mold for a period of 12 h to 72 h, more particularly 24 h to 48 h, and more particularly at a temperature of 18° C. to 30° C., preferably 20° C. to 25° C.

Further preferably, the process also includes a step c) of:
overmolding of additional elastomer material, more particularly by injection molding, onto the substrate provided with the at least one multilayer structure, more particularly the at least one multilayer structure transferred onto the substrate.

This advantageously allows the establishment of a final, i.e. definitive, shape of the substrate provided with the at least one multilayer structure, and thus of the product to be produced.

Preferably, step c) is (likewise) carried out in a forming tool or casting mold, more particularly in the forming tool or casting mold already mentioned.

With regard to further features and advantages of the process, reference is made in full to the statements made under the first aspect of the invention, which also apply mutatis mutandis to the process according to the second aspect of the invention.

According to a third aspect, the invention relates to a multilayer structure, more particularly a sensor or a sensor unit, including the following layers arranged on top of one another:
at least one metal layer and
at least one elastomer material layer.

With regard to further features and advantages of the multilayer structure, particularly in relation to the at least one metal layer and the at least one elastomer material layer and more particularly to an at least one adhesion layer optionally present, reference is made in full to the statements made in the above description, more particularly under the first aspect of the invention, which also apply mutatis mutandis to the multilayer structure according to the third aspect of the invention.

According to a fourth aspect, the invention relates to a process for producing a multilayer structure, more particularly according to the third aspect of the invention. The process includes the following steps:
a) applying at least one elastomer material layer onto a transfer film and
b) applying at least one metal layer onto the at least one elastomer material layer
or
a) applying at least one elastomer material layer onto a transfer film,
b) applying at least one adhesion layer onto the at least one elastomer material layer, and
c) applying at least one metal layer onto the at least one adhesion layer.

Preferably, the transfer film is applied to a carrier.

When performing step a), the at least one elastomer material layer may be applied directly or indirectly onto the transfer film. Preferably, the transfer film is covered or coated by the at least one elastomer material layer completely, i.e. continuously or over the entire surface area, when performing step a).

In addition, when carrying out step b), the at least one metal layer or at least one adhesion layer may be applied directly or indirectly onto the at least one elastomer material layer. The at least one elastomer material layer may in principle be covered or coated by the at least one metal layer or at least one adhesion layer completely or only in sections or regions. Preferably, the at least one metal layer or at least one adhesion layer covers or coats the at least one elastomer material layer only in sections or regions.

In addition, when carrying out step c), the at least one metal layer may be applied directly or indirectly onto the at least one adhesion layer. The at least one adhesion layer may in principle be covered or coated by the at least one metal layer completely or only in sections or regions. The at least one metal layer preferably covers or coats the at least one adhesion layer completely.

With regard to further features and advantages of the process, particularly in relation to the at least one metal layer, at least one elastomer material layer, and at least one adhesion layer, reference is made in full to the statements made in the above description, more particularly under the second aspect of the invention, which also apply mutatis mutandis to the process according to the fourth aspect of the invention.

Further features and advantages of the invention will become apparent from the following description of preferred embodiments with reference to figures and examples. Features of the invention may each be realized on their own or in combination with one another. The embodiments described hereinbelow serve to further elucidate the invention without the invention being limited thereto.

### BRIEF DESCRIPTION OF THE DRAWINGS

FIG. 1 shows an example of a functionalized metal-polymer multilayer composite structure.

FIG. 2 shows principal steps in a process.

FIG. 3 shows a process of transfer to a cylindrical PDMS base substrate.

FIG. 4 shows a finished PDMS cylinder with integrated sensor structures.

FIG. 5 shows a selected morphology region of a prepared specimen in relevant steps of an operation.

### DETAILED DESCRIPTION

### Examples Section

### 1. Experimental Methods

### 1.1 MPBC Sample Design

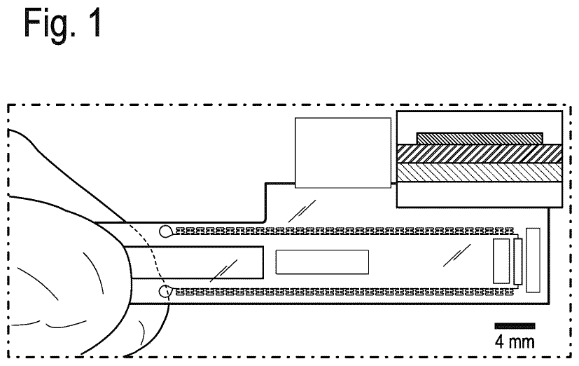

Metal-polymer multilayer composites, more particularly metal-polymer bilayer composites (MPBC), having a thin (approx. 100 μm) polydimethylsiloxane membrane (PDMS membrane) functionalized with structured 40 nm layers of Au were used. Two different structuring methods were used, one using conventional photolithographic processing and the other using an aperture mask. The Au metalization makes it possible for there to be stretch-sensitive regions and regions containing leads in which the resistance of said regions changes only slightly under stress. The leads here comprise self-similar serpentine structures. The sensor region employs a stretch-sensitive surface with microcracks. An example of a functionalized metal-polymer multilayer composite structure is shown in FIG. 1.

### 1.2 Substrate Preparation

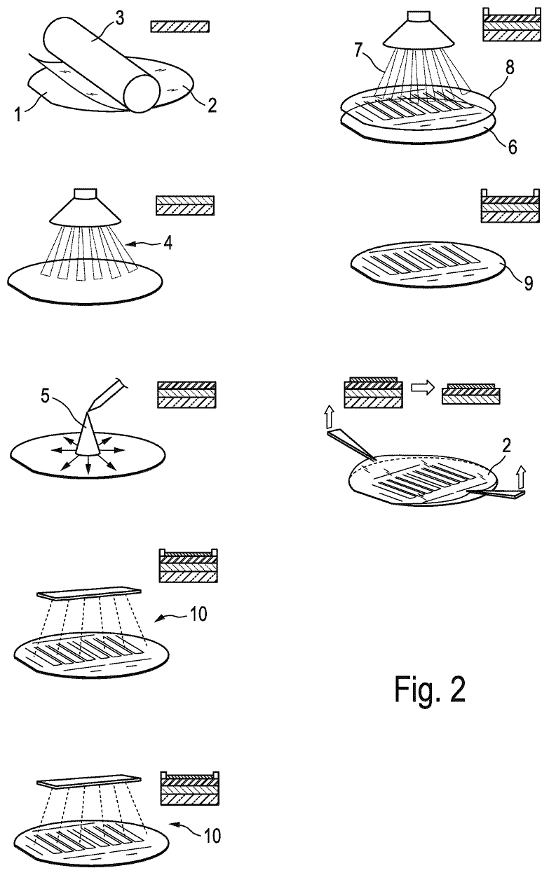

The principal steps in the process are shown in FIG. 2. 4-inch wafers 1 polished on one side and having a thickness of 0.5 mm were used as a handling platform. A UV-sensitive dicing film 2 (also known as a saw film) made of polyethylene terephthalate (PET) (Adwill D-203, 50 μm) was transferred onto the wafer 1 with a soft foam roller 3. The applied film 2 was cut with a scalpel along the wafer edges and then exposed to UV light 4 to reduce adhesion to the wafer 1 (special device having 5 Ster-L-Ray® low-pressure mercury lamps). At a minimum dose of about 160 mJ/cm², adhesion decreases to about 1% of its original value (according to the data sheet from 772.2 N/m to 5.8 N/m). To ensure a uniform and maximum reduction in adhesion, the UV activation was performed at an increased dose of about 250 mJ/cm² and was carried out under a nitrogen atmosphere to prevent ozone formation. After the UV treatment, a degassed, bubble-free PDMS prepolymer 5 (Elastomer kit marketed under the registered trademark SYLGARD 184®, mixture 10:1 (base to curing agent), mixed using a Thinky 250-ARE planetary centrifugal mixer) was applied onto the handling wafer 1 covered with the film 2 by spin coating at 600 rpm (acceleration 200 rpm/s) for 60 sec. A vacuum was then applied (10 min) to ensure a defect-free and even membrane, before it was cured in an oven at 90° C. for 30 min.

### 1.3 Structuring of the PDMS Membrane

In the case of photolithographic structuring, a negative lift-off photoresist 6 (marketed under the registered trademark AZ® nLOF 2070, Merck KGaA) was applied directly onto the PDMS surface by spin coating at 3000 rpm (acceleration 1000 rpm/s) for 60 sec. Soft baking at 100° C. for 2 min on a hotplate resulted in a photoresist thickness of about 6.5 μm. The exposure to light 7 with a chromium mask 8 employed a Suss MA6 mask aligner (350 W high-pressure mercury lamp) at a dose of 70 mJ/cm². The subsequent baking (post-exposure bake PEB) was carried out on a hotplate for 3 min at 115° C. To prevent cracks from forming in the stabilized photoresist layer as a result of the different thermal expansion coefficients of the layers making up the multilayer structure, the light-exposed wafers were immediately transferred to an oven preheated to 100° C. They were then allowed to cool slowly to room temperature over a period of about 5 h. For development, the AZ developer (Merck KGaA, undiluted, high-speed configuration) was prepared in a laboratory jar on an analog orbital shaker (RS-OS 5 Phoenix Instruments). The structures were developed for 1 min 50 sec with slight movement of the fluid and then rinsed with deionized water and dried under a stream of nitrogen.

In the case of structuring of the metal layer in an aperture mask process, a 50 μm nickel stencil mask 9 produced by applied microSWISS GmbH in a suitable UV-LIGA process was used. Since the mask had the outer dimensions of a standard 4-inch wafer, the mechanics of the Suss MA6 mask aligner were used to apply the mask onto the PDMS membrane surface by means of flat alignment and magnets. The surface-to-surface contact between the PDMS and the smooth mask surface ensured stable positioning through van der Waals interactions without the need for further fixation.

### 1.4 PVD Metal Deposition and Detachment

The vapor deposition 10 was carried out by thermal evaporation in an Edwards Auto 306 vacuum coating unit. To improve the adhesion of the 40 nm Au cover layer, an intermediate Ti layer of 4 nm was first deposited without any break in the vacuum (about 2*10⁻⁶ mbar). After the coating process, either the photoresist was dissolved in acetone with slight agitation of the fluid and the resulting multilayer system then rinsed with deionized water, or the aperture mask was gently pulled off the PDMS surface. The UV-activated stabilizing transfer film 2 could then be easily detached from the wafer 1 without exposing the thin functionalized PDMS membrane 6 to significant stress.

### 2. Transfer Process

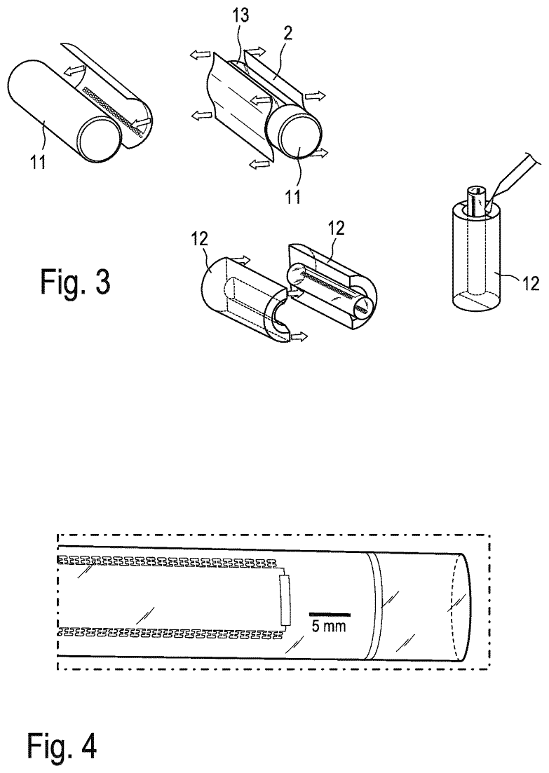

FIG. 3 shows the process of transfer to a cylindrical PDMS base substrate 11. The cylindrical base 11 was cast in a special polymethyl methacrylate (PMMA) mold having a constant outer diameter of 10 mm. The wall thickness was for the most part 400 μm and in specific regions only 200 μm. Since the specified transfer employed a connection of PDMS to PDMS via partial curing, it was in practice expedient to apply the intervening PDMS connecting layer by spin coating before detaching the film. PDMS-PDMS connection via partial curing permitted high connection strengths while being easy to execute. Here, a thin PDMS layer about 60 μm in thickness was applied onto the top side of the structured layer by spin coating at 900 rpm for 60 s (acceleration 200 rpm/s) and then cured at 50° C. for 45 min. This resulted in stabilization of the PDMS film while maintaining sticky properties comparable to an intermediate layer of adhesive. The relevant portion of the specimen was then precut with a scalpel, the stabilizing transfer film 2 was detached from the handling wafer with tweezers (see step 4 in FIG. 2), and the partially cured PDMS surface was attached to the appropriate position on the cylindrical base 11 (step 1 in FIG. 3). The specimen was then placed in a cylindrical PMMA mold 12 and left therein for 48 h at room temperature so that it cured completely. As a final step, the transfer film 2 was detached from the connected membrane layer 13 (FIG. 3, step 2), and a further casting process (FIG. 3, steps 3+4) and subsequent curing at room temperature for 48 h carried out so as to obtain a uniform cylindrical sensor.

### 3. Characterization and Results

To ensure that the transferred metal morphology is not altered by undefined stresses after the last step, it was necessary to remove the finished PDMS cylinder from the casting mold with due care. The inner part of the casting mold, which defined the variable wall thickness of the PDMS base cylinder, was therefore replaced by a water-soluble adhesive wax (2-M19 soluble stic wax, Paramelt B.V.) before the transfer process. For this, it was necessary for the subsequent curing steps of the connection process and the final casting steps to be carried out at low temperature in order to avoid melting the wax (drip melting point approx. 57° C.).

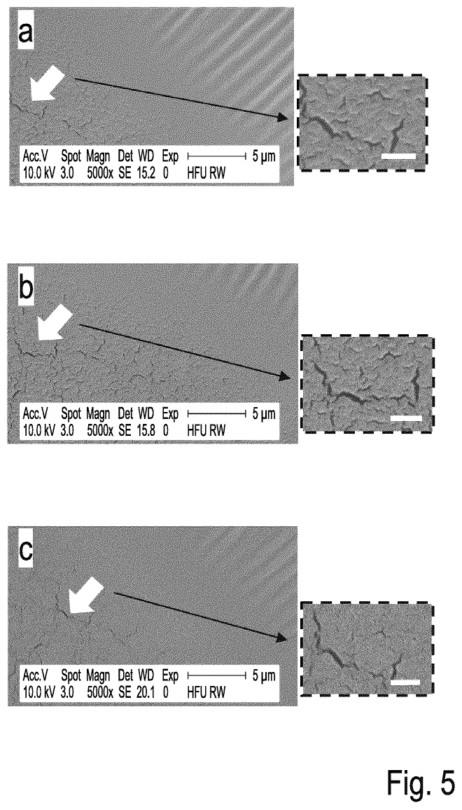

FIG. 4 shows the finished PDMS cylinder with the integrated sensor structures. As proof that the process permits the handling and transfer of fragile surface morphologies, various surfaces (with microcracks and corrugated intermittent structures) were introduced by selective plasma pretreatment and also by varying the deposition rate. To characterize the morphology in the initial state prior to connection, after detachment of the handling wafer, and in the final state on the cylindrical base, the same structure was investigated in an SEM (Philips XL30) at each step. To characterize the morphology after transfer to the cylindrical base, a special aluminum holder simulating the curvature of the PDMS base was used.

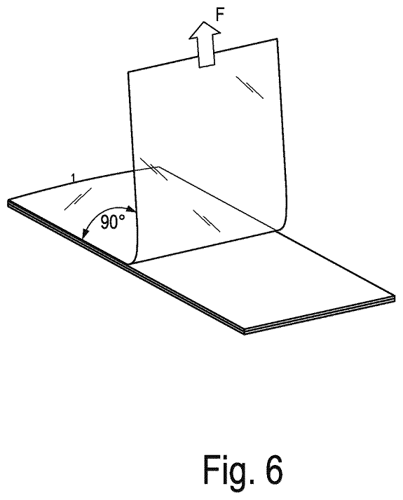

FIG. 5 shows a selected morphology region of the prepared specimen in each relevant step of the described operation. As can be seen, the fragile crack morphology was unchanged during handling of the membrane, the sole change being slight compression owing to the curvature of the cylindrical base (step c), while neither delamination nor crack expansion was observed. In addition, simple two-wire measurements of the resistance showed that the electrical integrity of the structure was maintained at about 4 kΩ.

Since there are various options for the process for connecting membrane to substrate, a 90° peeling test (see DIN EN 28510-1:2014) was carried out to determine the peeling force necessary to detach the cover film from the PDMS membrane (step 2, FIG. 3). For this purpose, specimens having a 100 μm PDMS membrane that had been produced according to the substrate preparation process described above (step 1, FIG. 2) were detached from the handling wafer and the PDMS surface connected to the polished side of precut wafer pieces (30 mm wide) after activation in an oxygen plasma (30 sec at 100 W). The transfer film was then attached to the load cell of the peeling test device and the tests were carried out at a constant speed of 50 mm/min and a peeling angle of 90°. Since the tests were limited by the size of the wafers and the width of the flats (wafers were cut perpendicular to the main flat close to the edges of the flat), the maximum peeling length was 90 mm and the peeling width used was 20 mm. A total of 5 samples between 20 mm and 80 mm peeling length were tested and analyzed to rule out edge effects. The average peeling force was determined to be 0.07 N/mm (SD 0.011 N/mm).

| No. | Peeling force [N/mm] |
|---|---|
| 1. | 0.076 |
| 2. | 0.076 |
| 3. | 0.082 |
| 4. | 0.078 |
| 5. | 0.051 |
| Ø | 0.07 |
| Standard deviation | 0.01 |

1. A medical device having at least one multilayer structure comprising:
at least one metal layer; and
at least one elastomer material layer.

2. The medical device according to claim 1, wherein the at least one elastomer material layer comprises an elastomer material selected from the group consisting of elastomers, thermoplastic elastomers, thermoplastic polyamide elastomers, thermoplastic copolyester elastomers, olefin-based thermoplastic elastomers, thermoplastic styrene block copolymers, urethane-based thermoplastic elastomers, olefin-based thermoplastic vulcanizates, olefin-based crosslinked thermoplastic elastomers, natural rubber vulcanizates, synthetic rubber vulcanizates, styrene-butadiene rubber, butadiene rubber, acrylonitrile-butadiene rubber, butyl rubber, ethylene-propylene-diene rubber, chloroprene rubber, polyisoprene rubber, polyalkylsiloxanes, polydimethylsiloxane, silicone rubbers, silicone elastomers, methyl silicone, vinyl methyl silicone, phenyl vinyl methyl silicone, phenyl-modified silicone, fluoroalkyl silicone, fluoro vinyl methyl silicone, and mixtures of at least two of the aforementioned elastomer materials.

3. The medical device ccording to claim 1, wherein the at least one metal layer includes at least one metal, selected from the group consisting of gold, platinum, indium, tin, copper, silver, gallium and alloys of at least two of the aforementioned metals.

4. The medical device according to claim 1, wherein the at least one elastomer material layer has a layer thickness of 0.0001 mm to 0.2 mm, and the at least one metal layer has a layer thickness of ≤150 nm.

5. The medical device according to claim 1, wherein the at least one metal layer directly covers the at least one elastomer material layer.

6. The medical device according to claim 1, wherein at least one adhesion layer is formed between the at least one metal layer and the at least one elastomer material layer.

7. The medical device according to claim 6, wherein the at least one metal layer covers the at least one adhesion layer directly and completely and the at least one adhesion layer covers the at least one elastomer material layer directly and only in regions.

8. The medical device according to claim 6, wherein the at least one adhesion layer includes at least one material selected from the group consisting of titanium, aluminum, chromium, and mixtures of at least two of the aforementioned materials.

9. The medical device according to claim 1, wherein the at least one multilayer structure further includes at least one additional elastomer material layer that directly covers the at least one metal layer.

10. The medical device according to claim 1, wherein the at least one multilayer structure or the at least one metal layer has a varying surface morphology.

11. The medical device according to claim 1, wherein the at least one multilayer structure forms part of a surface of the medical device or is integrated in a wall of the medical device.

12. The medical device according to claim 1, wherein the medical device is curved.

13. The medical device according to claim 1, wherein the medical device comprises a balloon catheter having a balloon that includes the at least one multilayer structure.

14. The medical device according to claim 1, wherein the at least one multilayer structure defines a sensor unit of the medical device.

15. A process for producing the medical device according to claim 1, the process comprising the steps of:
a) producing the at least one multilayer structure; and
b) transferring the at least one multilayer structure to a substrate, with formation of the substrate provided with the at least one multilayer structure.

16. A multilayer structure comprising:
at least one metal layers; and
at least one elastomer material layer.

17. The multilayer structure according to claim 16, wherein the multilayer structure comprises a sensor unit.
